(19) United States
(12) Patent Application Publication
(10) Pub. No.: US 2011/0190488 A1
(43) Pub. Date: Aug. 4, 2011

(54) Methods of Making Cyclic Amide Monomers and Related Derivatives

(76) Inventor: Douglas A. Wicks, Plymouth, MN (US)

(21) Appl. No.: 13/055,668

(22) PCT Filed: Jul. 24, 2009
(86) PCT No.: PCT/US2009/051753

§ 371 Date: Mar. 24, 2011

Related U.S. Application Data
(60) Provisional application No. 61/135,835, filed on Jul. 24, 2008.

(51) Int. Cl. C07D 223/12; C07D 201/08; C07D 201/16

(52) U.S. Cl. 540/528; 540/538

(57) ABSTRACT

The present invention relates to methods of making a cyclic amide. The methods include the step of heating a fermentation broth in a manner effective to produce a cyclic amide, wherein the fermentation broth includes an amino acid or salt thereof. The cyclic amide monomers can be polymerized in a manner effective to form a polyamide. One advantage of the present invention is that lysine and/or salt thereof can be heated to form α-amino-ε-caprolactam while the lysine is still in the fermentation broth. The lysine and/or salt thereof do not need to be purified from the fermentation broth prior to being heated to form α-amino-ε-caprolactam. For example, the fermentation broth does not need to be subjected to an ion exchange process prior to being heated to form α-amino-ε-caprolactam. Avoiding such an ion exchange process can substantially reduce manufacturing costs.

### BACKGROUND

One method of making ε-caprolactam includes using benzene as a starting chemical compound, which can be converted to either cyclohexane or phenol and either chemical can be converted via cyclohexanone to cyclohexanone oxime and then this intermediate can be heated in sulfuric acid. This chemical reaction is known as the Beckman rearrangement. The starting chemical benzene can be produced via the refinement of a non-renewable source of petroleum.

A sugar such as a non-toxic glucose is an alternative source for making ε-caprolactam. In order to use glucose as a replacement for benzene as a starting point for many of these syntheses, a bio-refinery can be used. A bio-refinery is a facility that integrates biomass conversion processes and equipment to produce fuels, power and chemicals from biomass. The bio-refinery concept is analogous to a petroleum refinery which produces multiple fuels and products from petroleum. By producing multiple products, a bio-refinery can take advantage of the differences in biomass components and intermediates and maximize the value derived from the biomass feed stock with minimal waste and emissions. The conversion of biomass into a sugar such as glucose is well known in the art (see Advancing Sustainability Through Green Chemistry and Engineering, ACS Symposium Series, 823, edited by Lanky, R. L. and Anastas, P. T., American Chemical Society, Washington, D.C., 2002; Biomass for Energy, Industry and Environment, 6^{th} European Community Conference, edited by Grassi, G., Collina, A. and Zibetta, H., Elsevier Science Publishing Co., Inc., New York, 1998; Biobased Industrial Products: Research and Commercialization Priorities, edited by Dale, B. E., Natural Research Council, Washington, D.C., 1999; Emerging Technologies for Materials and Chemicals from Biomass, ASC Symposium 467, edited by Narayan, R., Rowell, R., Schultz, T., American Chemical Society, Washington, D.C., 1991).

Bacterial fermentation which starts with a sugar and produces lysine is known. L-lysine is produced and available from many industrial sources including such companies as Aginomoto, Kyowa Hakko, Sewon, Archer Daniels Midland, Cheil Jedang, BASF, and Cargill.

The cyclization of L-lysine to form a seven member ring of α-amino-ε-caprolactam has been attempted before and reports have shown low yields. Such attempts have included reactions in near super critical water (see Japanese Patent No. 2003206276 to Goto et al. issued Jul. 22, 2003) or reactions using an excess of Al₂O₃ in toluene (see Blade-Font, A., Tetrahedron Lett., 1980, 21, 2443-2446. Pellegata, R., Pinza, M.: Pifferi G., Synthesis 1978, 614-616).

U.S. Pub. No. 2007/0149777 (Frost), discloses a method of making α-amino-ε-caprolactam from lysine, converting α-amino-ε-caprolactam to ε-caprolactam, and making nylon 6 from ε-caprolactam.

### BRIEF DESCRIPTION OF THE DRAWINGS

The advantages of the present invention, and the manner of attaining them, will become more apparent and the invention itself will be better understood by reference to the following description of the embodiments of the invention taken in conjunction with the accompanying drawing, wherein:

FIG. 1—is a block diagram of a method according to the present invention for making cyclic amide monomers and a polyamide, according to the present invention, from a fermentation broth.

### DETAILED DESCRIPTION

The embodiments of the present invention described below are not intended to be exhaustive or to limit the invention to the precise forms disclosed in the following detailed description. Rather the embodiments are chosen and described so that others skilled in the art may appreciate and understand the principles and practices of the present invention. While the present invention will be described in the specific context of using lysine to ultimately make ε-caprolactam monomers, the principles of the invention are applicable to other amino acids in a fermentation broth and other cyclic amide monomers as well.

The present invention includes methods of making cyclic amide (also referred to herein as “lactam”) monomers from a fermentation broth.

As used herein, a “fermentation broth” is a product of fermentation, where the fermentation process produces one or more amino acids, and/or salts thereof. An amino functional carboxylic acid useful in the invention can cyclize to form a stable lactam, preferably a lactam having from 5 to 8 ring members. An amino functional carboxylic acid useful in the invention can contain other functional groups as long as those functional groups do not interfere with the amidation reaction (e.g., an amidation reaction optionally mediated by an alcohol solvent (discussed below)). In certain embodiments, the one or more amino acids include at least lysine and/or a salt thereof. Lysine is an amino acid that can be produced via fermentation and has the chemical formula C₆H₁₄N₂O₂. Lysine that is produced via fermentation can include isomers of lysine such as structural isomers, stereoisomers, and combinations of these. Structural isomers of lysine include α-lysine and β-lysine. A “structural” isomer of lysine means that one of the amino groups is located at a different position along the carbon chain. For example, α-lysine can be represented by the following chemical structure:

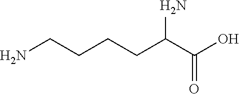

Whereas β-lysine can be represented by the following chemical structure:

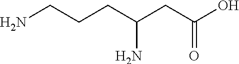

Each of α-lysine and β-lysine isomers can have stereoisomers such as L-α-lysine, D-α-lysine, L-β-lysine, and D-β-lysine. The L and D isomers of lysine are optical isomers (enantiomers) meaning that the L and D isomers are mirror images of each other but the L and D isomers cannot be superimposed onto each other. In preferred embodiments, lysine includes at least L-lysine. Specific forms of lysine include, e.g., L-lysine dihydrochloride, L-lysine hydrochloride, L-lysine phosphate, L-lysine diphosphate, L-lysine acetate, L-lysine sulfate, and L-lysine, combinations of these, and the like.

In addition to including one or more amino acids, and/or salts thereof, a fermentation broth includes one or more other products of fermentation. For example, a fermentation broth can include fermentation microorganisms, sugars, salts, lipids, protein fragments, combinations of these, and the like. Preferably, the cellular material is separated from the extracellular material prior to forming a lactam from the amino acid precursor. The cellular material includes the microorganisms used for fermentation. The extracellular material includes material in the fluid that is outside the plasma membranes of the fermentation microorganisms. For example, the extracellular material can include metabolites, ions, proteins, one or more amino acids, and/or salts thereof, sugars, salts, lipids, and protein fragments. Separating the cellular material from extracellular material can include inactivating and filtering the fermentation microorganisms from the extracellular material. Preferably, a fermentation broth according to the present invention has not been subjected to a process that isolates one type of amino acid(s) and/or salt(s) thereof (e.g., lysine or a salt thereof) from different amino acid(s) and/or salt(s) thereof that are present after fermentation. In embodiments where the fermentation broth includes lysine and/or a salt thereof, the fermentation broth preferably includes at least one amino acid and/or salt there in addition to the lysine and/or salt thereof. Preferably, the fermentation broth that is heated to form a lactam from the amino acid and/or salt thereof also includes other products of fermentation (e.g., metabolites, ions, proteins, sugars, salts, lipids, protein fragments, combinations of these, and the like).

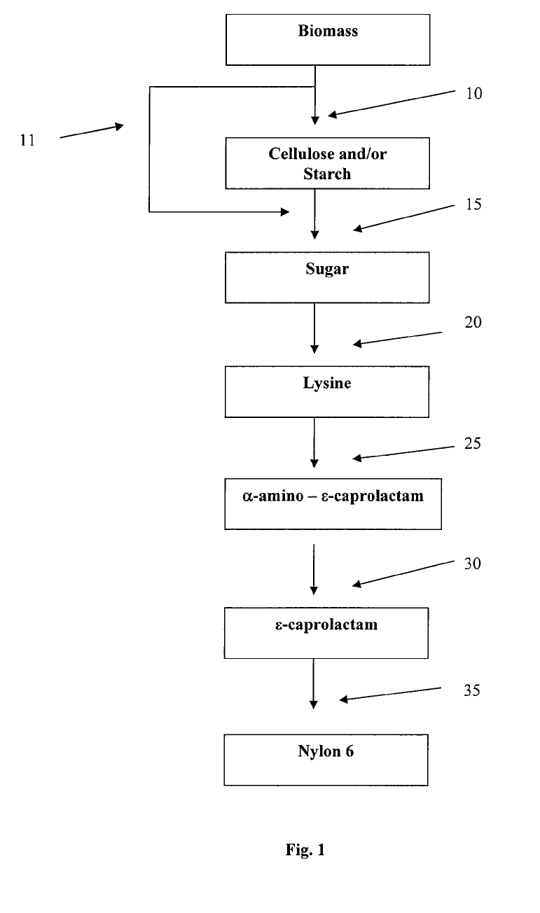

Advantageously, e.g., in the context of FIG. 1, the lysine and/or salt thereof can be heated to form α-amino-ε-caprolactam while the lysine is still in the fermentation broth and in the presence of one or more additional amino acids and/or other products of fermentation. The lysine and/or salt thereof do not need to be purified from the fermentation broth prior to being heated to form α-amino-ε-caprolactam. For example, the fermentation broth does not need to be subjected to an ion exchange process prior to being heated to form α-amino-ε-caprolactam. Avoiding such an ion exchange process can substantially reduce manufacturing costs.

Any fermentation broth that includes at least an amino acid that can be formed into a lactam can be used in a method of the present invention. Methods of making fermentation broths are well-known. See, e.g., U.S. Pub. No. 2007/0149777 (Frost) and Savas Anastassiadis, “L-Lysine Fermentation,” Recent Patents on Biotechnology 2007, volume 1, pages 11-24, Bentham Science Publishers Ltd. (2007), the entireties of which references are incorporated herein by reference. An exemplary fermentation broth for use with the present invention is described in U.S. Pat. No. 5,840,358 (Höfler et al.), the entirety of which is incorporated herein by reference. A method of making L-β-lysine in particular is described in international publication number WO 2007/101867 (Zelder et al.), the entirety of which is incorporated herein by reference. An exemplary fermentation broth for use in the present invention is commercially available under the trade name Biolys® from Evonik Degussa Corporation, Kennesaw, Ga.

Starting materials for microbial fermentation for use in the present invention are well-known. Such materials include bacteria and nutrients for the bacteria such as biomass, polyol (e.g., glycerol), combinations of these, and the like. Referring to FIG. 1, a new process is shown for the cyclization of L-lysine to α-amino-ε-caprolactam, which is ultimately converted into nylon 6. As shown, biomass is ultimately converted to sugar. Biomass is a material produced by the growth of microorganisms, plants or animals, is supplied to the system. Examples of a biomass include agricultural products and by-products such as corn, husks, stalks, cereal crops, alfalfa, clover, grass clippings, vegetable residues, straw, maize, grain, grape, hemp, sugar cane, flax, and potatoes; forestry and paper products and by-products such as sawdust paper, cellulose, wood pulp, wood chips, pulp sludge and leaves, combinations of these, and other appropriate materials that are known in the art. The biomass can be high cellulose-containing materials, high starch-containing materials, and combinations of these. As shown in FIG. 1 by step 10, in some embodiments, the biomass can be fractionated yielding such components as cellulose, hemicellulose, lignocellulose, plant oil, and/or starch. The block labeled “Cellulose and/or Starch” may include starch, cellulose, hemicellulose, lignocellulose, or combinations thereof and the like. Such separation or fractionization of biomass into cellulose components and/or starch is well known in the art (see, e.g., U.S. Pat. No. 6,022,419 to Torget et al. issued Feb. 8, 2000; U.S. Pat. No. 5,047,332 to Chahal issued Sep. 10, 1991; U.S. Pat. No. 6,228,177 to Torget issued May 8, 2001; U.S. Pat. No. 6,620,292 to Wingerson issued Sep. 16, 2003; and B. Kamm and M. Kamm, Biorefinery-Systems, Chem. Biochem. Eng. Q. 18 (1) 1-6 2004). In alternative embodiments, as shown by step 11, the biomass is not separated but, rather, the biomass moves directly to step 15.

In step 15 of FIG. 1, cellulose components, starch, or combinations thereof are converted to a sugar such as glucose by hydrolysis. In various embodiments, the box labeled “Sugar” may include but is not limited to glucose, dextrose, xylose, sucrose, fructose, arabinose, glycerol, other sugars or polyols known to one skilled in the art or combinations thereof and the like. In various embodiments of the invention, the raw biomass is converted to a sugar by hydrolysis. In various embodiments of the invention, the hydrolysis is an acid hydrolysis. In other embodiments of the invention, the hydrolysis is enzymatic hydrolysis. Methods of hydrolysis that can produce a sugar such as glucose are well known in the art (see U.S. Pat. No. 6,692,578 to Schmidt et al. issued Feb. 17, 2004, U.S. Pat. No. 5,868,851 to Lightner issued Feb. 9, 1999, U.S. Pat. No. 5,628,830 to Brink issued May 13, 1997, U.S. Pat. No. 4,752,579 to Arena et al. issued Jun. 21, 1988, U.S. Pat. No. 4,787,939 to Barker et al. issued Nov. 29, 1988, U.S. Pat. No. 5,221,357 to Brink issued Jun. 22, 1993 and U.S. Pat. No. 4,615,742 to Wright issued Oct. 7, 1986). Depolymerization of hemicellulose can produce D-xylose and L-arabinose, which can serve as alternative starting materials for microbial synthesis of chemicals. Plant oils are another component of biomass. Transesterification of plant oils leads to esterified fatty acids which can be used as biodiesel and glycerol, which is another polyol suitable for use as a starting material in microbial synthesis. In various embodiments of the invention, step 15 may produce other sugars that may or may not include glucose.

Fermentation of L-lysine produced from sugars such as glucose is known. The Corynebacterium glutamicum bacterium is able to synthesize lysine. Through classical strain optimization, the bacteria have become able to synthesize large quantities of lysine. Production can take place in fermenters in which the Corynebacterium glutamicum bacterium converts raw sugars such as glucose, sugar cane, and/or molasses into lysine. Such processes are well known in the art (see U.S. Pat. No. 2,979,439 to Kinoshita et al. issued Apr. 11, 1961, U.S. Pat. No. 3,687,810 to Kurihara et al. issued Aug. 29, 1972, U.S. Pat. No. 3,707,441 to Shiio et al. issued Dec. 26, 1972, U.S. Pat. No. 3,871,960 to Kubota et al. issued Mar. 18, 1975, U.S. Pat. No. 4,275,157 issued to Tosaka et al. issued Jun. 23, 1981, U.S. Pat. No. 4,601,829 issued to Kaneko issued Jul. 22, 1986, U.S. Pat. No. 4,623,623 issued to Nakanishi et al. issued Nov. 18, 1986, U.S. Pat. No. 4,411,997 issued to Shimazaki et al. issued Oct. 25, 1983, U.S. Pat. No. 4,954,441 issued to Katsumata et al. issued Sep. 4, 1990, U.S. Pat. No. 5,650,304 issued to Ishii et al. issued Jul. 22, 1997, U.S. Pat. No. 5,250,423 issued to Murakami et al. issued Oct. 5, 1993, U.S. Pat. No. 4,861,722 issued to Sano et al. issued Aug. 29, 1989, and Manufacturing of Stabilised Brown Juice for L-lysine Production—from University Lab Scale over Pilot Scale to Industrial Production, M. H. Thomsen et al., Chem. Biochem. Eng. Q. 18 (1) 37-46 (2004)).

According to the present invention, the fermentation broth is heated in a manner effective to produce cyclic amide monomers. In preferred embodiments, cyclic amide monomers have ring sizes in the range from 5 to 8 ring members. In certain embodiments, cyclic amide monomers made according to the present invention include caprolactams such as α-amino-ε-caprolactam, β-amino-ε-caprolactam, ε-caprolactam, and combinations of these. Referring to FIG. 1, step 25 shows reaction cyclization of α-lysine in the fermentation broth to α-amino-ε-caprolactam. An α-amino-ε-caprolactam monomer can be represented by the following chemical structure (I):

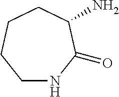

If β-lysine is present in the fermentation broth, the cyclization reaction of β-lysine produces β-amino-ε-caprolactam which is represented by the following chemical structure:

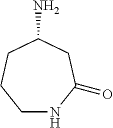

In various embodiments, water that is generated during the cyclization reaction may or may not be removed from the reaction. One exemplary method of removing water from the reaction includes using a Dean-Stark trap. Other methods known within the art may be used to remove the water such as evaporation, crystallization, distillation or any other appropriate method known by one skilled in the art. In various embodiments of the invention, water is removed as an azeotrope.

The cyclization reaction may be performed using L-Lysine sulphate in the presence of its byproducts from fermentation either as a spray dried mass or as an aqueous mixture as found before drying and as described in U.S. Pat. No. 5,840,358 (Höfler et al.), the entirety of which is incorporated herein by reference.

Optionally, the cyclization reaction may be performed using catalysts as described in U.S. Pub. No. 2007/0149777 (Frost), the entirety of which is incorporated herein by reference. In some embodiments of the invention, the catalyst is aluminum oxide (Al₂O₃).

The cyclization reaction may be performed following free basing as described in U.S. Pub. No. 2007/0149777 (Frost), the entirety of which is incorporated herein by reference, or without free basing in conjunction with the addition of a small amount of a strong acid such as HCl.

Optionally, the step of heating the fermentation broth comprises heating the fermentation broth in the presence of a solvent including an alcohol. Using an alcohol in the cyclization reaction can be performed in a manner as described in U.S. Pub. No. 2007/0149777 (Frost), the entirety of which is incorporated herein by reference.

Exemplary alcohols include aliphatic mono-ols or diols. In some embodiments of the invention, the alcohol has about 2 to about 6 carbons. Non-limiting examples of alcohols include 1-propanol, 2-propanol, 1-butanol, 2-butanol, isobutanol, 1,2-propanediol, 1,3-propanediol, 1,2-butanediol, 1,4-butanediol, all isomers of 5 carbon monols, diols and triols including with out limitation 1-pentanol, 1,2-pentanediol, 1,5-pentanediol, and all isomers of 6 carbon monodiols, diols and triols including without limitation, 1-hexanol, 1,2-hexanediol, 1,6-hexanediol. Other non-limiting examples of 2 to 6 carbon alcohols include glycerol, trimethylolpropane, pentaerythritol and the like. In various embodiments, the alcohols have a single hydroxyl group. In other embodiments, the alcohols have 2 hydroxyl groups. In some embodiments, the alcohols have 3 hydroxyl groups. Non-limiting examples of glycols include propylene glycol, butylene glycol, neopentyl glycol and the like. In a preferred embodiment of the invention, the alcohol is 1,2-propanediol. In addition to the higher yields by the use of the 1,2-propanediol, this organic alcohol may be readily available at a bio-refinery since it may be obtained by the hydrogenation of lactic acid which may be readily available as a co-product produced from the biomass.

In various embodiments of the invention, neutralized L-lysine can be heated in an alcohol. In various embodiments of the invention, the heating of the neutralized L-lysine in the alcohol can be accomplished by reflux. In various embodiments of the invention, the heating of the alcohol and the neutralized lysine in the presence of a catalyst can accomplished by reflux.

The following are some non-limiting examples based on reaction (1).

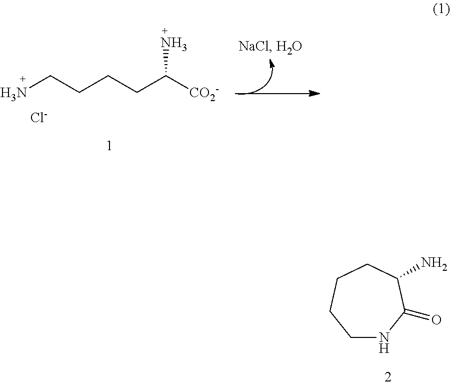

The temperature of the cyclization reaction can be similar to that described in U.S. Pub. No. 2007/0149777 (Frost), the entirety of which is incorporated herein by reference. In various embodiments, the heating is at a high enough temperature to allow azeotropic removal of water with the alcohol. In various embodiments, the heating is below a temperature that polymerizes the caprolactam. In some embodiments, the heating is at temperatures from about 99° C. to about 201° C. One exemplary method of heating to form cyclic amide monomers includes contacting the fermentation broth with steam in a manner effective to form cyclic amide monomers. Preferably, steam is used to contact a fermentation broth that is in the form of a spray dried mass as described in U.S. Pat. No. 5,840,358 (Höfler et al.).

Optionally, an amino group can be removed (known as deaminating) from cyclic amide monomers (e.g., the α-amino group can be removed from the α-amino-ε-caprolactam in a manner effective to produce ε-caprolactam). Step 30 of FIG. 1 shows the deaminization of α-amino-ε-caprolactam to ε-caprolactam. An ε-caprolactam monomer can be represented by the following chemical structure II:

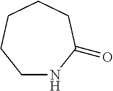

Methods for deaminating organic compounds are well known in the art. Deamination processes can be chosen depending on the reaction conditions, yield, and/or cost.

One preferred method of deamination includes contacting α-amino-ε-caprolactam, or salt thereof, with a catalyst and a gas that includes hydrogen gas in a manner that removes the α-amino group and provides ε-caprolactam. Optionally, the step of contacting can be performed in the presence of a solvent. Such a method is described in International Publication No. WO 2008/103366 by Frost, the entire disclosure of which is incorporated herein by reference.

In various embodiments, deamination may be accomplished by reacting the amino functional intermediate with hydroxylamine-O-sulphonic acid and KOH catalysts. The hydroxylamine-O-sulphonic acid (NH₂OSO₃H) may be prepared by the reaction of bis(hydroxylammonium sulfate ((NH₂OH)₂H₂SO₄) with fuming sulphuric acid (H₂SO₄—SO₃) (see Matsuguma et al., Inorg. Syn. 1957, 5, 122-125). In certain embodiments of the invention, the deamination reaction is run after the removal of NaCl after the completion of the cyclization reaction as described above. Deamination reactions using hydroxylamine-O-sulphonic acid have been described before but have produced low yields of ε-caprolactam (see Doldouras, G. A., Kollonitsch, J., J. Am. Chem. Soc. 1978, 100, 341-342; Ramamurthy, T. V., Ravi, S., Viswanathan, K. V. J. Labelled Compd. Rad., 1987, 25, 809-815). In accordance with the present invention, the reaction temperature is lowered to below the freezing point of water during the addition of the hydroxylamine-O-sulphonic acid. In various embodiments of the invention, the temperature is lowered to about −5° C., and in other embodiments, the temperature is lowered to about −20° C. In various embodiments, the amine is washed away with a solvent. The solvent may be water or a mixture of water and a small organic alcohol. In various embodiments of the invention, the solvent is water.

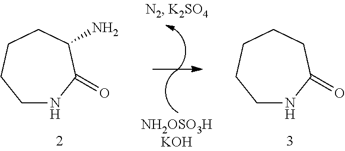

Following cyclic amidation, other reactive groups besides an amino group on the cyclic ring may be removed if desired.

Optionally, in various embodiments of the process as described in FIG. 1, additions may be made such that the amine that is a by-product from step 30 may be recycled so that the nitrogen may be added in step 20 as a nutrient for fermentation. In other optional embodiments, the amine that is a by-product in step 30 may be recycled so that the nitrogen may be added in step 15 as a nutrient for fermentation. Optionally, one skilled in the art may precipitate the monophosphate or diphosphate salt of lysine. The sodium phosphate salt (monobasic or dibasic) generated during cyclization of lysine phosphate maybe (like ammonia above) from step 30 may be recycled so that the phosphorus may be added in step 20 as a nutrient for fermentation.

Optionally, in various embodiments of the invention, a portion of the biomass may be converted into lactic acid and then hydrogenated into 1,2-propanediol which maybe used in Step 25. The process of taking biomass and converting it into lactic acid is well known in the art. (See U.S. Pat. No. 6,403,844 to Zhang et al. issued Jun. 11, 2002, U.S. Pat. No. 4,963,486 to Hang issued Oct. 16, 1990, U.S. Pat. No. 5,177,009 issued Kampen issued Jan. 5, 1993, U.S. Pat. No. 6,610,530 issued to Blank et al. issued Aug. 26, 2003, U.S. Pat. No. 5,798,237 issued to Picataggio et al. issued Aug. 25, 1998, and U.S. Pat. No. 4,617,090 to Chum et al. issued Oct. 14, 1986, Zhang, Z; Jackson, J. E.; Miller, D. J. Appl. Catal. A-Gen. 2001, 219, 89-98, Zhang, Z; Jackson, J. E.; Miller, Ind. Eng. Chem. Res. 2002, 41, 691-696).

Cyclic amide monomers made according to the present invention can be polymerized in a manner effective to form a polyamide. For example, the ε-caprolactam monomers that can be used to make polyamides which can be used in the manufacture of synthetic fibers, especially nylon 6 that is also used in carpet fibers, bristle brushes, textile stiffeners, film coatings, synthetic leather, plastics, plasticizers, vehicles, and cross linking for polyurethanes. Preferably, the cyclic amide monomers are isolated from the fermentation broth before polymerizing.

The production of nylon 6 is shown as step 35 and can be accomplished by the ring opening polymerization of the monomer ε-caprolactam. The polymerization reaction is a ring opening polymerization from the monomer ε-caprolactam which can be accomplished by heating the ε-caprolactam to about 250° C. with about 0.3% to about 10% water present. See U.S. Pat. No. 2,142,007 to Schlack issued Dec. 27, 1938 and U.S. Pat. No. 2,241,321 to Schlack issued May 6, 1941. The polymerization of ε-caprolactam to nylon 6 is well known in the art. A non-limiting example of such polymerization is as follows: nylon 6 may be produced by hydrolytic polymerization of ε-caprolactam, with predominant use of a VK tube (abbreviation for the German expression “vereinfacht Kontinuierlich” which means simplified continuous) a heated vertical flow pipe. The molten ε-caprolactam, with 0.3-5% of water, chain length regulators, and, if necessary, a dulling agent, can be fed from above, and the polymer melt is discharged at the reactor bottom. Typically the VK tube is equipped with 3 heat exchangers establishing the temperature profile along the reactor. The VK-tube consists of a plug flow zone in the lower part and a mixing/evaporating zone in the top. The function of the top part is to heat up the reaction mass and to evaporate excess water thus setting the total water content in the polymer melt. The endothermic ε-caprolactam ring opening reaction is started, followed by exothermal polyaddition and polycondensation. With the central heat exchanger, the temperature is corrected and equalized over the tube cross section. After passing the central heat exchanger, the temperature rises to about 270-280° C. due to the heat of reaction. The bottom heat exchanger drops the temperature to 240-250° C., thus reaching a higher degree of polymerization in the equilibrium. Simultaneously a higher degree of ε-caprolactam conversion to nylon 6 can be achieved. Specifically designed inserts can be applied evening out the dwell time over the tube cross section. Sixteen to twenty hours may be the mean dwell time in the tube. Relative solution viscosities from 2.4 to 2.8 are achieved with a single stage process (solvent: 96% sulphuric acid, concentration: 1 g/100 ml, temperature: 25° C.). The maximum capacity may be 130 tonnes/day. In the 2-stage technology, a prepolymerizer, operated under pressure and with high water content, can be followed by a final VK polymerizer operated at atmospheric pressure or vacuum. The high reaction rate of the ε-caprolactam ring opening under the conditions in the prepolymerizer yields a low total residence time making the process suitable for very high throughput rates up to 300 tonnes/day.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention.

1-7. (canceled)

8. A process for synthesizing α-amino-ε-caprolactam, the process comprising the step of heating a mixture comprising a fermentation broth and an alcohol, without the presence of a catalyst, at a temperature of about 99° C. to about 250° C. to produce α-amino-ε-caprolactam, wherein the fermentation broth comprises lysine.

9. (canceled)

10. A process according to claim 8, wherein the alcohol has from 2 to 6 carbons.

11. A process according to claim 10, wherein the alcohol comprises a diol, a triol, and/or a glycol.

12-13. (canceled)

14. A process according to claim 10, wherein the alcohol is from the group consisting of ethanol, 1-propanol, 1-butanol, 1-pentanol, 1-hexanol, 1,2-propanediol, and mixtures thereof.

15. A process according to claim 10, wherein the alcohol is 1,2-propanediol.

16. A process according to claim 8, wherein the heating is below the temperature of polymerization of ε-caprolactam.

17. A process according to claim 8, wherein the heating allows removal of water.

18. (canceled)

19. A process for the synthesis of ε-caprolactam, the process comprising:
a) heating a mixture comprising a fermentation broth and an alcohol at a temperature of about 99° C. to about 250° C. to produce α-amino-ε-caprolactam, wherein the fermentation broth comprises lysine; and
b) deaminating the α-amino-ε-caprolactam by a method comprising contacting the α-amino-ε-caprolactam at least once with a deamination catalyst in a manner effective to remove the α-amino group and provide ε-caprolactam.

20. A process according to claim 19, wherein the yield of ε-caprolactam is greater than about 70%.

21. (canceled)

22. A process according to claim 19, wherein the temperature in step (b) is from about −5° C. to about −20° C.

23. A process according to claim 19, wherein the process further comprises removing an amine, produced by the deaminating step (b), using a washing step.

24-25. (canceled)

26. A process according to claim 19, wherein the heating step (a) comprises heating in the presence of a catalyst.

27. A process according to claim 26, wherein the catalyst is Al₂O₃.

28. A process according to claim 19, wherein the alcohol has from 2 to 6 carbons.

29. A process according to claim 28, wherein the alcohol is selected from the group consisting of ethanol, 1-propanol, 1-butanol, 1-pentanol, 1-hexanol, 1,2-propanediol, and mixtures thereof.

30. A process according to claim 19, wherein the alcohol is 1,2-propanediol.

31. A process according to claim 19, wherein the heating step (a) is below the temperature of polymerization of ε-caprolactam.

32. A process according to claim 19, wherein the heating allows removal of water.

33. (canceled)

34. A process according to claim 19, wherein the deaminating step (b) includes a hydrodenitrogenation catalyst.

35-55. (canceled)

56. A process according to claim 34, wherein the deaminating step (b) further includes contacting the α-amino-ε-caprolactam with a hydrogen gas to remove the α-amino group in the presence of the hydrodenitrogenation catalyst.
